(19) United States
(12) Patent Application Publication
(10) Pub. No.: US 2026/0108381 A1
(43) Pub. Date: Apr. 23, 2026

(54) MANDIBULAR ADVANCEMENT DEVICE WITH AT LEAST ONE PROTRUSION

(71) Applicant: DCSTAR INC, New York, NY (US)

(72) Inventor: David LUO, New York, NY (US)

(21) Appl. No.: 18/924,663

(22) Filed: Oct. 23, 2024

(51) Int. Cl. A61F 5/56

(52) U.S. Cl. CPC A61F 5/566

(57) ABSTRACT

A mandibular advancement device includes an upper tray assembly and a lower tray assembly. The upper tray assembly includes an upper moldable component and an upper frame, and the lower tray assembly includes a lower moldable component and a lower frame. The device has a terminal point near the lingual side, and near the terminal point, at least one protrusion is provided on the upper frame and the lower frame. The at least one protrusion is configured to allow the patient's mouth to open and close and to maintain the relative front-to-back position of the upper frame and the lower frame, thereby allowing the device to keep the patient's airway open by advancing the mandible.

### TECHNICAL FIELD

The present disclosure relates to a mandibular advancement device with at least one protrusion, particularly to a device used to prevent, reduce, or eliminate snoring and/or obstructive sleep apnea.

### BACKGROUND

Mandibular Advancement Devices (MADs) are among the most common dental orthotics used to treat respiratory-related disorders, similar to mouth guards. They are frequently employed to treat Obstructive Sleep Apnea Syndrome (OSA). By physically advancing the mandible (lower jaw), these devices increase the cross-sectional area of the upper airway, thereby maintaining airway openness during sleep, preventing airway collapse and obstruction, and improving respiratory conditions. The device functions by accommodating the upper and lower dental arches, with the wearer maintaining the device in position during sleep. By misaligning and securing the upper and lower parts of the device, the patient's lower jaw is held in a position that is more forward than its usual relaxed state. This adjustment allows the airflow to pass smoothly through the patient's pharyngeal pathway, thereby reducing or eliminating occurrences of snoring and/or sleep apnea.

Mandibular advancement devices provide a non-surgical treatment option for respiratory diseases, offering a simple and risk-free alternative to surgical treatments. Compared to CPAP machines, MADs are more compact and portable, facilitating use in various settings and enhancing patient compliance. For patients who find CPAP machines uncomfortable, MADs are a suitable alternative.

Existing mandibular advancement devices on the market are categorized into custom and non-custom models. Custom MADs are tailor-made based on dental impressions to fit perfectly within a patient's oral structure, offering better comfort and stability. However, these custom devices necessitate personalized tailoring, that is, taking an oral impression and then custom-making a mandibular advancement device that fits the patient perfectly, and require dental professional involvement and supervision, making them more costly and time-consuming to produce. Non-custom MADs, on the other hand, are designed in standard sizes, which may not fit all oral structures precisely, potentially causing discomfort for some users. Due to their mass production, non-custom MADs are more cost-effective and readily available for immediate use without the need for dental professional intervention, saving both time and money for the patient.

Patients have different options available, but for those with mild symptoms, limited budgets, or who desire immediate treatment, non-custom mandibular advancement devices offer a more economical choice. These devices can also serve as an initial or transitional treatment option.

### SUMMARY

This disclosure aims to provide a mandibular advancement device that is convenient for patients to use, reduces their costs, and is comfortable to wear.

In one embodiment, a mandibular advancement device with at least one protrusion is provided. The mandibular advancement device with at least one protrusion includes an upper tray assembly and a lower tray assembly. The upper tray assembly is configured to be positioned adjacent to an upper dental arch of a patient when placed in an oral cavity, while the lower tray assembly is configured to be positioned adjacent to a lower dental arch of the patient when placed in the oral cavity. The upper tray assembly includes an upper moldable component and an upper frame. The upper moldable component and the upper frame are configured to interconnect during use and to form at least one press fit when the patient bites down, and a required deformation temperature of the upper moldable component, upon heating, is lower than that of the upper frame. The lower tray assembly includes a lower moldable component and a lower frame. The lower moldable component and the lower frame are configured to interconnect during use and to form at least one press fit when the patient bites down, and a required deformation temperature of the lower moldable component, upon heating, is lower than that of the lower frame. The upper frame and the lower frame each have side portions configured to be near cheeks during use, and at least one of the side portions of the upper frame and the lower frame has the at least one protrusion. The at least one protrusion is configured to allow a mouth of the patient to open and close and to maintain the front-to-back positional relationship of the upper frame and lower frame.

In one embodiment, the upper moldable component and the lower moldable component are configured in an arched shape, and the arched shape includes at least one of an oval arch, a conical arch, or a square arch.

In one embodiment, the upper frame and the lower frame each have a bottom wall that is configured to be opposite an occlusal surface of teeth when the patient bites down, and at least part of the bottom wall of the upper frame is configured to contact the bottom wall of the lower frame.

In one embodiment, the upper moldable component and the lower moldable component include a flexible thermoplastic material.

In one embodiment, the hardness of a material of the upper frame is at least partially greater than the hardness of a material of the upper moldable component, and the hardness of a material of the lower frame is at least partially greater than the hardness of a material of the lower moldable component.

In one embodiment, the upper tray assembly and the lower tray assembly each have a contour that is higher at a middle and lower at sides nearer the cheeks.

In another embodiment, a mandibular advancement device with at least one protrusion is provided. The mandibular advancement device with at least one protrusion includes an upper tray assembly and a lower tray assembly. The upper tray assembly has an arched contour configured to conform to a curvature of an upper dental arch of a patient, and the lower tray assembly has an arched contour configured to conform to a curvature of a lower dental arch of the patient. The upper tray assembly includes an upper moldable component and an upper frame. The upper moldable component and the upper frame are configured to interconnect during use, and a required deformation temperature of the upper moldable component, upon heating, is lower than that of the upper frame. The lower tray assembly includes a lower moldable component and a lower frame. The lower moldable component and the lower frame are configured to interconnect during use, and a required deformation temperature of the lower moldable component, upon heating, is lower than that of the lower frame. The upper frame and the lower frame each have side portions configured to be near cheeks during use, and at least one of the side portions of the upper frame and the lower frame has the at least one protrusion. The at least one protrusion is configured to allow a mouth of the patient to open and close and to maintain the front-to-back positional relationship of the upper frame and lower frame. The at least one protrusion has one or more of the following characteristics: (a) a height of the at least one protrusion between 1.7 mm to 22.5 mm; (b) a distance between the at least one protrusion and other parts of the upper frame and/or lower frame being less than 16.5 mm in a top view; and (c) the upper frame and the lower frame having front ends configured to be near labial sides during use, and a distance between the at least one protrusion and the front ends being greater than 10 mm.

In one embodiment, the upper frame has at least one groove to accommodate the upper moldable component.

In one embodiment, the lower frame has at least one groove to accommodate the lower moldable component.

In one embodiment, the upper moldable component and the lower moldable component are configured to soften when heated in hot water such that the upper moldable component and the lower moldable component are adjusted and molded to a shape that is configured to conform to the upper dental arch and the lower dental arch, respectively, when the patient bites down.

In one embodiment, the upper moldable component and the lower moldable component are made of a moldable material, with the required deformation temperature upon heating being less than 100 degrees Celsius.

In one embodiment, the upper frame and the lower frame are made of a material with high thermal stability, with the required deformation temperature upon heating being greater than 100 degrees Celsius.

In yet another embodiment, a mandibular advancement device with at least one protrusion is provided. The mandibular advancement device with at least one protrusion includes an upper tray assembly and a lower tray assembly. The upper tray assembly is configured to be positioned adjacent to an upper dental arch of a patient when placed in an oral cavity, and the lower tray assembly is configured to be positioned adjacent to a lower dental arch of the patient when placed in the oral cavity. The upper tray assembly includes an upper moldable component and an upper frame. The upper moldable component and the upper frame are configured to interconnect during use and to form at least one press fit when the patient bites down, and a required deformation temperature of the upper moldable component, upon heating, is lower than that of the upper frame. The lower tray assembly includes a lower moldable component and a lower frame. The lower moldable component and the lower frame are configured to interconnect during use and to form at least one press fit when the patient bites down, and a required deformation temperature of the lower moldable component, upon heating, is lower than that of the lower frame. The upper frame and the lower frame each have side portions configured to be near cheeks during use, and at least one of the side portions of the upper frame and the lower frame has the at least one protrusion. The at least one protrusion is configured to allow a mouth of the patient to open and close and to maintain the front-to-back positional relationship of the upper frame and lower frame, and the at least one protrusion forms at least one arc.

In one embodiment, the upper frame remains in contact with the lower frame without separating during use.

In one embodiment, the upper moldable component and the lower moldable component are made of a medical-grade moldable material.

In one embodiment, the upper moldable component and the lower moldable component allow for slight deformation while retaining their deformed shape.

In one embodiment, the upper frame and the lower frame each have an outer wall, and the upper moldable component and the lower moldable component each at least partially cover a top of the outer wall, respectively.

In a further embodiment, a mandibular advancement device with at least one protrusion is provided. The mandibular advancement device with at least one protrusion includes an upper tray assembly and a lower tray assembly. The upper tray assembly has an arched contour configured to conform to a curvature of an upper dental arch of a patient, while the lower tray assembly has an arched contour configured to conform to a curvature of a lower dental arch of the patient. The upper tray assembly includes an upper moldable component and an upper frame. The upper moldable component and the upper frame are configured to interconnect during use and to form at least one press fit when the patient bites down, and a required deformation temperature of the upper moldable component, upon heating, is lower than that of the upper frame. The lower tray assembly includes a lower moldable component and a lower frame. The lower moldable component and the lower frame are configured to interconnect during use and to form at least one press fit when the patient bites down, and a required deformation temperature of the lower moldable component, upon heating, is lower than that of the lower frame. The upper frame and the lower frame each have side portions near the cheeks during use, and at least one of the side portions of the upper frame and the lower frame has the at least one protrusion. The at least one protrusion is configured to allow a mouth of the patient to open and close and to maintain the front-to-back positional relationship of the upper frame and lower frame. The mandibular advancement device has a terminal point that is configured to be away from a labial side during use, with the distance between the at least one protrusion on the upper frame and the terminal point being shorter than the distance between the at least one protrusion on the lower frame and the terminal point.

In one embodiment, the upper frame has an outer wall, and the at least one protrusion on the upper frame conforms to a curvature of the outer wall of the upper frame.

In one embodiment, the lower frame has an outer wall, and the at least one protrusion on the lower frame conforms to a curvature of the outer wall of the lower frame.

In one embodiment, a height at a middle portion of the upper frame and the lower frame is greater than a height at the terminal point of the mandibular advancement device.

In one embodiment, the upper frame and the lower frame each have a bottom wall that is configured to be opposite an occlusal surface of teeth during use, and a connection point of the at least one protrusion with the upper frame and/or the lower frame is flush with the bottom wall of the upper frame and/or the lower frame.

The implementation of a mandibular advancement device provided by this disclosure has at least the following beneficial effects:
1. Traditional thermoplastic mandibular advancement devices, based on general feedback, still have many areas for improvement. In particular, most thermoplastic devices have a fixed structure that does not accommodate the need for patients to open their mouths to breathe during treatment. Although some mandibular advancement devices have added at least one protrusion on the bottom walls of the upper frame and/or the lower frames to create a breathing passage, this method does not allow patients to freely control the range of mouth opening and closing. This also can be inconvenient for patients who prefer to keep their mouths closed during treatment. To overcome this limitation, this disclosure introduces an innovative and improved design for the mandibular advancement device, enabling it to be adjustable to any angle within a certain range for both open and closed mouth conditions. This is achieved by placing at least one protrusion on at least one side portion near the cheeks on the upper frame and/or the lower frames, replacing the traditional form of snap-fitting or hinge connections at the bottom walls of the frames. This design not only enhances the user experience by allowing patients to naturally perform daily activities while wearing the device, but also significantly improves treatment compliance and patient satisfaction.
2. This disclosure, through careful design improvements, offers a mandibular advancement device that is more comfortable for the patient. Extensive research has been conducted to address various challenges, resulting in a device that enhances comfort during use. Since the device needs to fit closely with the human body, it requires careful attention to ergonomics and a design process informed by extensive human data, making it a complex and challenging project. For instance, the device must stay securely in place against the patient's upper dental arch during use. The height and width of the at least one protrusion are specifically designed to allow the patient to comfortably open and close their mouth with minimal impact. The design of the at least one protrusion is such that they continuously restrict the patient's lower jaw during the opening and closing of the mouth (i.e., maintaining the effect of mandibular advancement), ensuring the upper frame and the lower frame of the device stay connected without separating from each other while the mouth moves. These improvements were developed through targeted design solutions and extensive testing. The details of the upper frame and the lower frame, as well as the moldable components have been optimized to ensure they fit the dental arches more closely. The upper tray assembly, in particular, is designed to fit snugly with the upper dental arch and stay in place, preventing detachment due to gravity even when the mouth is open. The height of the at least one protrusion is set between 1.7 mm and 22.5 mm to allow for a wide mouth opening without pressing against the patient's oral structure. The distance between the at least one protrusion and the front ends is set to be more than 10 mm. Placing the at least one protrusion closer to the terminal point of the device can increase the opening angle between the upper frame and the lower frame but may also increase the chance of contact with the patient's oral tissues, possibly causing discomfort. Therefore, a balance is needed between these factors. The at least one protrusion on the upper frame and/or the lower frames needs to engage at any angle within a certain range, so they are designed with a slight outward offset. This offset helps ensure the effectiveness of the treatment and improves patient comfort. Additionally, the contact point between the at least one protrusion on the upper frame and the lower frame includes at least one arc. This design ensures smooth operation during the opening and closing of the mouth, reducing friction and avoiding jamming. These design features are not just simple structural choices; they are the result of precise calculations and optimizations. To achieve the best results, extensive experimental testing and data recording were necessary. Through repeated trials and fine adjustments, the shapes and dimensions of the at least one protrusion were gradually refined. Extensive data analysis and feedback were used to optimize the design of the at least one protrusion and other aspects, ensuring that every detail has been rigorously tested and optimized to guarantee patient comfort and the durability of the device.
3. This disclosure offers a more cost-effective alternative that achieves the same results as customized mandibular advancement devices. In the current market, only customized devices can effectively achieve the desired mouth opening and closing function. These custom devices are tailored under the guidance of professional physicians, ensuring a perfect fit with the patient's teeth and oral structure, which provides a high level of comfort. However, because they are custom-made, they require advanced manufacturing equipment and a fully digital manufacturing process, making them expensive—often costing thousands of dollars, which can be a significant financial burden for patients. In contrast, this disclosure integrates at least one protrusion into a non-custom mandibular advancement device, along with the use of upper and lower moldable components and frames, and precise control over various parameters of the device. This design allows the device to achieve the same mouth opening and closing effect as custom devices. It is a more suitable option for patients with limited budgets, offering an affordable treatment solution without compromising the device's effectiveness or comfort. This improvement makes the device accessible to a wider range of patients, achieving a better balance between personalization and cost-effectiveness. Additionally, this mandibular advancement device can be produced quickly, reducing the waiting time associated with custom devices, making it a better choice for patients seeking faster treatment.
4. The mandibular advancement device provided by this disclosure not only expands the scenarios in which it can be used, enhancing patient comfort, but also makes treatment more stable and extends the product's lifespan. a. The mandibular advancement device provided by this disclosure meets various needs in a patient's daily life, allowing them to engage in activities such as speaking and drinking while wearing the device, without the need to use the upper and lower dental arches to forcefully clamp and hold the device in place in the mouth. Most patients who require mandibular advancement devices suffer from snoring, particularly those who snore due to throat issues, where the mouth involuntarily opens and closes with each breath. Traditional devices with rigid structures make it difficult to breathe naturally and perform other oral activities while wearing them. The mandibular advancement device provided by this disclosure reduces the psychological burden on patients, adapts to the needs of different individuals, and offers a more ideal treatment option. b. The design of this mandibular advancement device carefully considers the complexity of the oral structure, ensuring comfort through precise calculations. Devices with rigid fixation can restrict oral movement, potentially causing discomfort or even pain during use. The inclusion of at least one protrusion in this design allows for a certain degree of freedom in oral movement, significantly improving comfort. c. Additionally, the upper and lower moldable components are made of flexible materials, further enhancing the safety and comfort of the device for the teeth. Typically, the main body of a custom-made mandibular advancement device (the part that directly contacts the teeth) is made from a single rigid material. In contrast, this disclosure features flexible materials that come into contact with the inside of the mouth, avoiding pressing against the oral tissues and reducing the sensation of a foreign object in the mouth during use. Moreover, the flexible material provides cushioning in the event of an accidental impact, offering protection to the teeth and making this a safer mandibular advancement device. d. The at least one protrusion in the mandibular advancement device provided by this disclosure allows it to be used in a variety of situations, increasing the product's stability and making it less likely to dislodge during use. As a result, patients can use this device for longer periods, reducing interruptions in treatment caused by device movement or other issues, and ensuring better adherence to the treatment regimen. e. The at least one protrusion also ensures a stable fit between the upper and lower tray assemblies and the patient's upper and lower dental arches, even when the mouth is open. This reduces fatigue from continuously clenching the device, meaning patients do not need to replace the device frequently. This feature is highly beneficial, contributing to increased patient satisfaction.

### BRIEF DESCRIPTION OF THE DRAWINGS

FIG. 1 is an overview diagram of the human respiratory system, including the nasal cavity, oral cavity, larynx, esophagus, trachea, and bronchi;

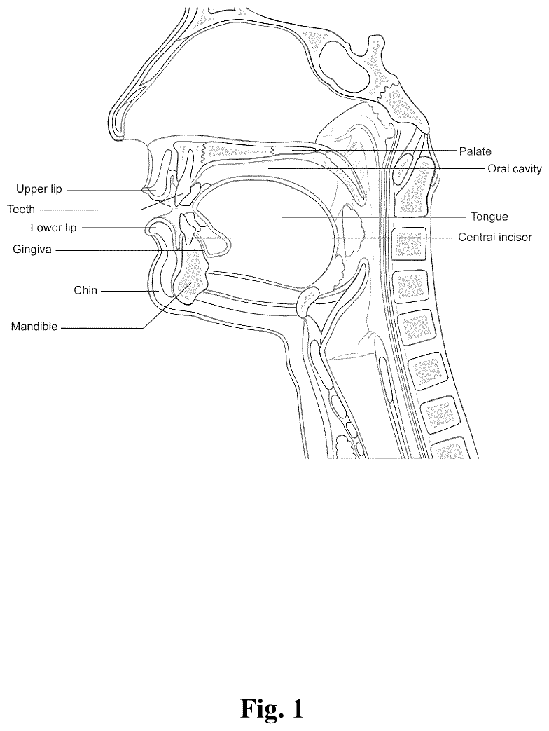

FIG. 2 is an anatomical overview diagram of the human oropharyngeal region, showing several identified surface anatomical features, including the oral cavity, pharynx, cervical vertebrae, salivary glands, and mandible;

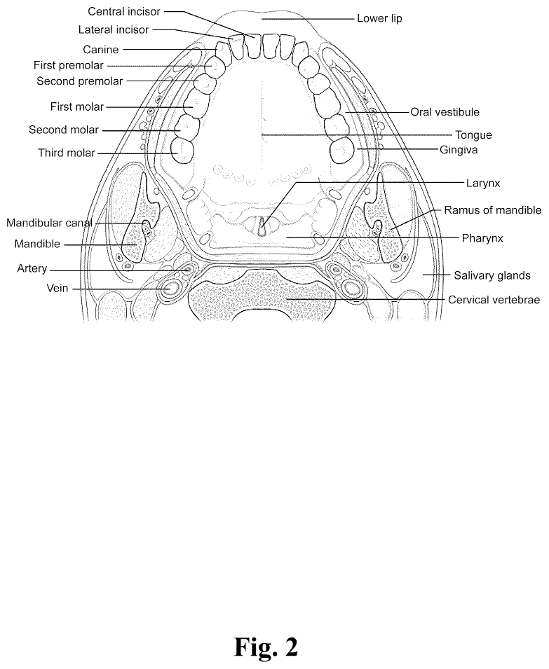

FIG. 3 is an overview diagram of the interior of the human oral cavity, showing several identified surface anatomical features, including teeth, palate, pharynx, tongue, and gingiva;

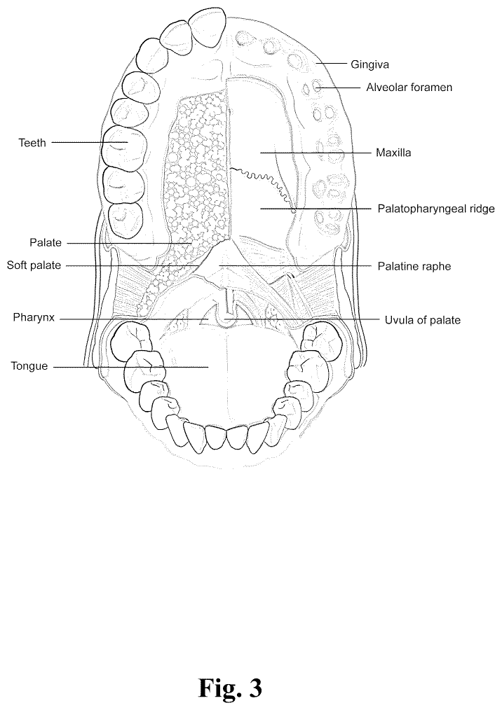

FIG. 4 is a side view of the human skull, including the mandible, teeth, vertebrae, larynx, maxilla, and condyle;

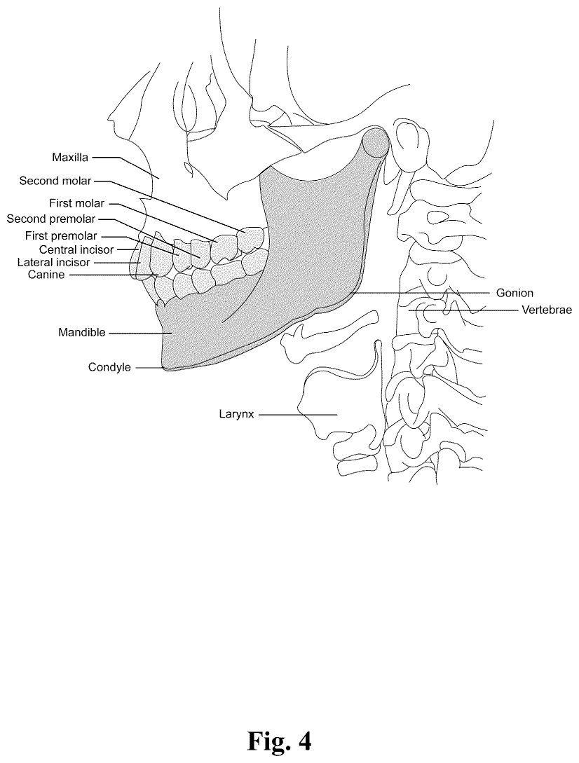

FIG. 5 is a side view of the human skull and oral muscles, including the buccinator muscle, orbicularis oris muscle, labial part of orbicularis oris muscle and marginal part of orbicularis oris muscle;

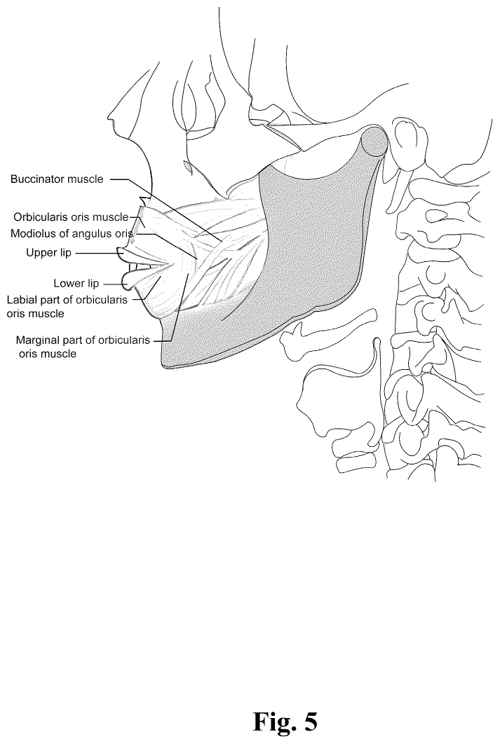

FIG. 6 is a side view of the mandibular structure in the head, showing the approximate position of the upper dental arch and mandible;

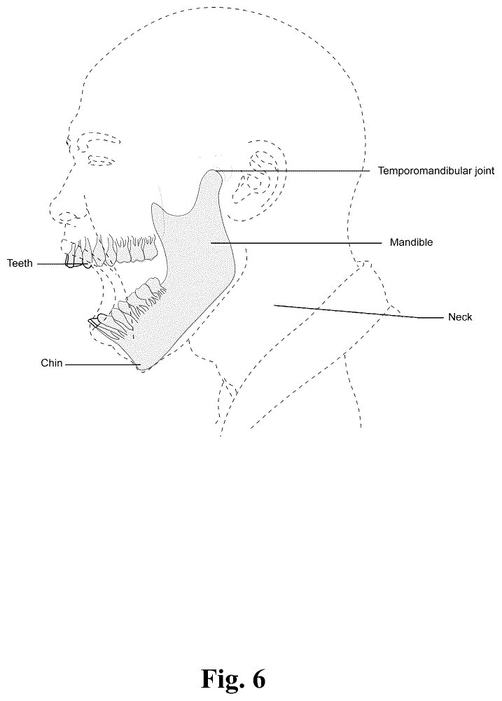

FIG. 7 is a schematic diagram of the specific structure of the mandible, including the teeth, chin, neck of mandible, condylar process, and mandibular condyle;

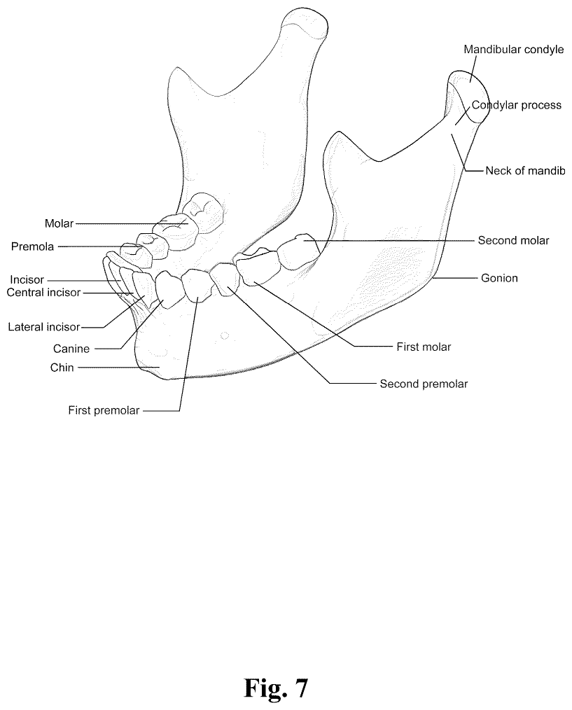

FIG. 8 is an anatomical overview diagram of the mandible, including the teeth, body of the mandible, superior mental spines, and mandibular torus;

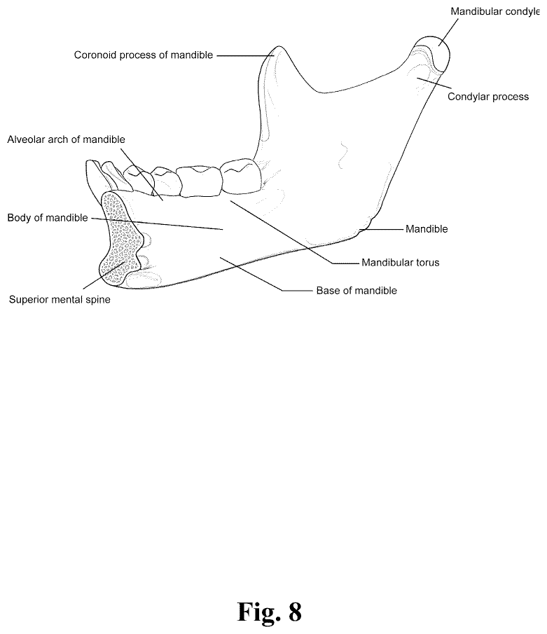

FIG. 9 is a schematic diagram of the movement of the mandible relative to the maxilla, showing the rotation and movement of the mandible with the condyle as the pivot point;

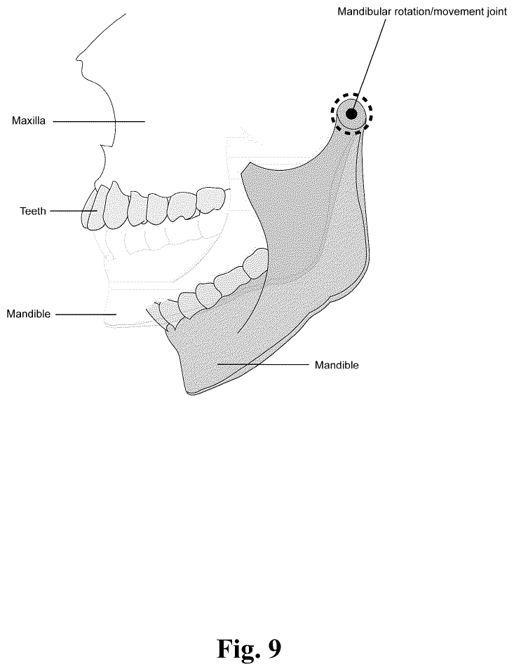

FIG. 10 is an overall schematic diagram of the mandibular advancement device in accordance with one embodiment;

FIG. 11 is an exploded structural diagram of the mandibular advancement device in accordance with one embodiment;

FIGS. 12A, 12B and 12C are a top view of the shapes of the upper tray assembly and the upper dental arch in accordance with multiple embodiments;

FIG. 13 is a schematic diagram of the engagement between the upper moldable component and the upper frame in accordance with one embodiment;

FIG. 14 is a three-dimensional schematic diagram of the upper frame and the lower frame with at least one protrusion in accordance with one embodiment;

FIG. 15 is a three-dimensional schematic diagram of the mandibular advancement device with at least one protrusion in the closed position in accordance with one embodiment;

FIG. 16 is a three-dimensional schematic diagram of the mandibular advancement device with at least one protrusion in the open position in accordance with one embodiment;

FIG. 17 is a schematic diagram of the engagement between the upper frame and the lower frame in accordance with one embodiment;

FIG. 18 is a top view of the mandibular advancement device with at least one protrusion in accordance with one embodiment;

FIG. 19 is a schematic diagram of the opening mechanism of the mandibular advancement device in accordance with one embodiment;

FIGS. 20A, 20B and 20C are schematic diagrams of the engagement between the at least one protrusion of the upper frame and the lower frame in accordance with one embodiment;

FIG. 21 is a schematic diagram of the engagement between the at least one protrusion and the upper and/or the lower frame in accordance with one embodiment.

FIG. 22 is a top view of the upper frame and/or the lower frame in accordance with one embodiment, where the protrusion has the same curvature as the outer wall of the frame;

FIG. 23 is a top view of the upper frame and/or the lower frame in accordance with one embodiment, where the protrusion has a different curvature from the outer wall of the frame;

FIG. 24 is a schematic diagram of the various measurements of the protrusion of the upper and/or lower frame in accordance with one embodiment;

FIG. 25 is a schematic diagram of the positioning of the protrusion of the upper frame and/or the lower frame in accordance with one embodiment;

FIG. 26 is a schematic diagram of an alternative form of the protrusion in accordance with one embodiment;

FIG. 27 is a three-dimensional schematic diagram of the upper frame or the lower frame with at least one protrusion in accordance with one embodiment;

FIG. 28 is an exploded schematic diagram of the upper frame or the lower frame with at least one protrusion in accordance with one embodiment.

### DETAILED DESCRIPTION

To elucidate the objectives, features, and advantages of this disclosure more clearly, a detailed description of specific embodiments of the disclosure is provided below in conjunction with the accompanying drawings. Many specific details are provided in this description to facilitate a comprehensive understanding of the disclosure. However, it is feasible to implement the disclosure in various ways other than those described here, and one skilled in the art can make similar modifications without departing from the essence of the disclosure. Therefore, the disclosure is not confined to the specific embodiments disclosed herein.

Compared to existing mandibular advancement devices on the market, this disclosure, through the design and use of at least one protrusion on the upper frame and/or the lower frames, allows for a greater range of oral movement while maintaining treatment effectiveness during sleep. This enhances the comfort and convenience of using the device, while also improving its effectiveness to some extent, ensuring sustained, effective treatment during sleep. As a result, this mandibular advancement device achieves better performance in terms of effectiveness, comfort, reliability, and durability. Not only does it improve performance metrics, but it also provides patients with a more stable, safe, and comfortable experience.

This disclosure offers a mandibular advancement device 1 that is easy for patients to use, enhances convenience, and provides comfort during wear.

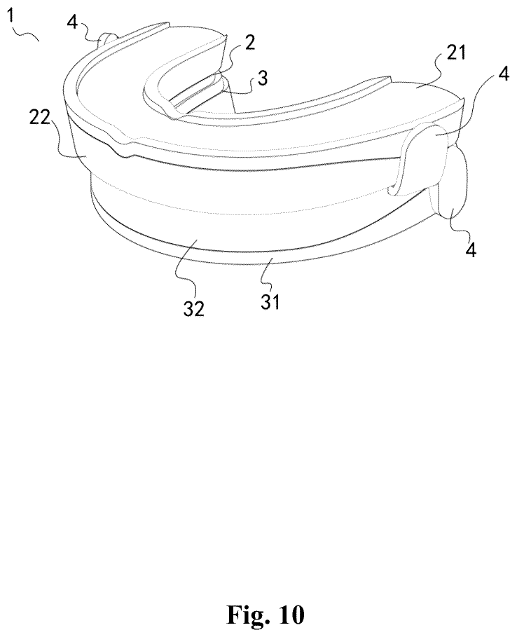

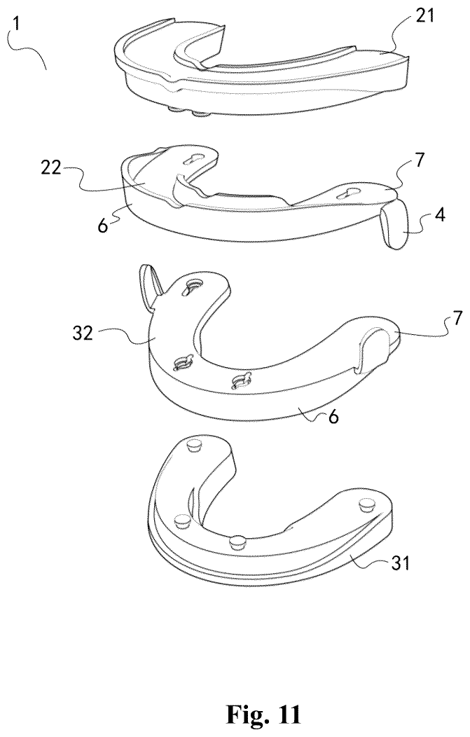

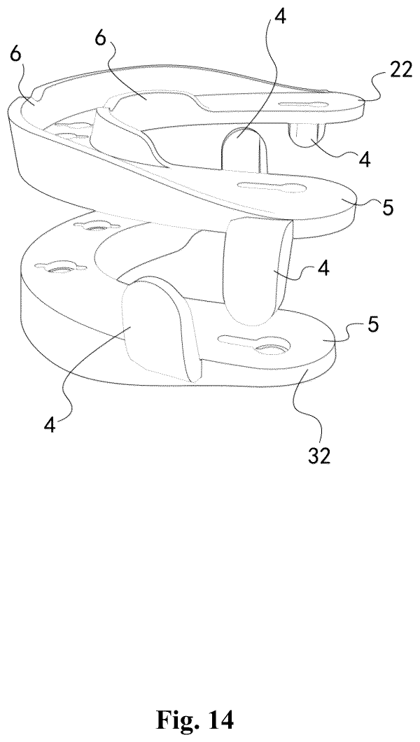

Specifically, as shown in FIGS. 10 to 27, the mandibular advancement device 1 of this disclosure includes an upper tray assembly 2 and a lower tray assembly 3. The upper tray assembly 2 has an arched contour that conforms to the curve of the patient's upper dental arch and is configured to be positioned adjacent to the patient's upper dental arch when placed in the oral cavity. The lower tray assembly 3 also has an arched contour that conforms to the curve of the patient's lower dental arch and is configured to be positioned adjacent to the patient's lower dental arch when placed in the oral cavity. Both the upper tray assembly 2 and the lower tray assembly 3 each have a contour that is higher at a middle and lower at the sides nearer the cheeks to accommodate the corresponding structure of the oral cavity. The upper tray assembly 2 and the lower tray assembly 3 are typically two separate parts. The mandibular advancement effect is achieved by moving the relative position of these two parts, specifically by shifting the lower tray assembly 3 towards the labial side relative to the upper tray assembly 2 and fixing it in place. The upper tray assembly 2 is fixed to the patient's upper dental arch, while the lower tray assembly 3 is fixed to the patient's lower dental arch. This configuration pulls the patient's mandible towards the labial side, thereby achieving the therapeutic effect.

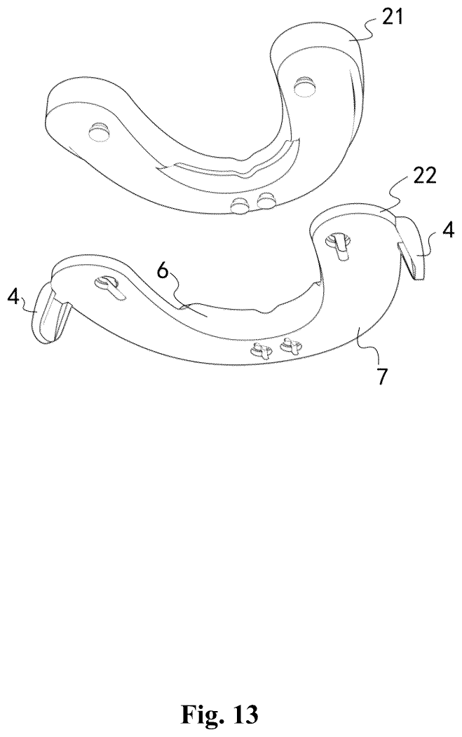

The upper tray assembly 2 includes an upper moldable component 21 and an upper frame 22. The upper moldable component 21 and the upper frame 22 are configured to connect and engage with each other during use, achieving at least one press fit when the patient bites down. Similarly, the lower tray assembly 3 includes a lower moldable component 31 and a lower frame 32. The lower moldable component 31 and the lower frame 32 are configured to connect and engage with each other during use, achieving at least one press fit when the patient bites down (as shown in FIG. 13, which only illustrates the fit between the upper moldable component 21 and the upper frame 22). The upper frame 22 and the lower frame 32 each have an outer wall 6 and a bottom wall 7. These connections can take various forms, including detachable or fixed, non-detachable connections. In the case of detachable connections, the connection between the upper moldable component 21 and the upper frame 22, as well as between the lower moldable component 31 and the lower frame 32, may involve protrusion and groove connections, snap-fit connections, clamping connections by the outer wall 6, or additional components such as pins or magnets. Non-detachable connections may involve screws, pins, or similar fasteners. To enhance the stability and portability of the mandibular advancement device 1, it is preferred that the connections between the upper moldable component 21 and the upper frame 22, as well as between the lower moldable component 31 and the lower frame 32, be either snap-fit connections or clamping connections by the outer wall 6. Typically, in snap-fit connections, the upper moldable component 21 and the lower moldable component 31 have at least one protrusion that aligns with corresponding grooves on the upper frame 22 and the lower frame 32. The at least one protrusion and corresponding grooves can be positioned in the middle section (i.e., the part of the mandibular advancement device 1 nearer the patient's incisors) and/or symmetrically positioned at both ends (i.e., the part of the device 1 nearer the patient's temporomandibular joint). When the upper moldable component 21 and the upper frame 22, as well as the lower moldable component 31 and the lower frame 32, are connected by clamping through the outer wall 6, both the upper frame 22 and the lower frame 32 have outer walls 6 on the labial and lingual sides. These outer walls 6, along with the bottom wall 7, form grooves that securely hold the upper moldable component 21 and the lower moldable component 31 in place. Specifically, the upper frame 22 has a groove that accommodates the upper moldable component 21, and the lower frame 32 has a groove that accommodates the lower moldable component 31.

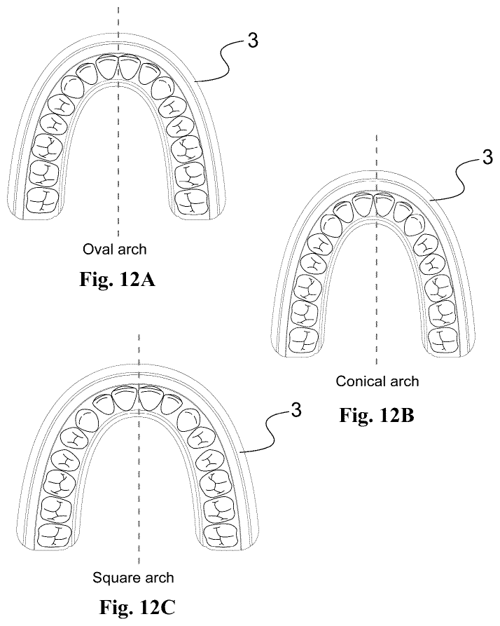

The upper moldable component 21 and the lower moldable component 31 are configured to at least partially conform to the teeth and/or gingiva. Since the arched shape of the upper and lower dental arches can generally be categorized into three types: oval arch, conical arch, and square arch, the upper moldable component 21 and the lower moldable component 31 are designed with an arched shape, specifically adopting at least one of the elliptical, conical, or square arch shapes (as shown in FIGS. 12A, 12B and 12C). To ensure patient comfort, the upper moldable component 21 and the lower moldable component 31 are made of a flexible thermoplastic material that is designed to soften when heated in hot water, allowing them to be adjusted and molded to match the patient's upper and lower dental arches during biting. The hot water used should be at least 60 degrees Celsius. The flexible material helps reduce irritation and abrasions to the soft tissues in the mouth. To ensure the effectiveness of the upper moldable component 21 and the lower moldable component 31, the flexible material should have a weight that is equal to or greater than 3.3 grams. The process of fitting the mandibular advancement device 1 to the patient's mouth involves heating and softening the material of the upper moldable component 21 and the lower moldable component 31, then biting down to mold them into the desired shape. Once the upper moldable component 21 and the lower moldable component 31 have taken on the deformed shape, they allow for slight adjustments to fine-tune the fit. To maintain the overall therapeutic effectiveness of the mandibular advancement device 1, the upper moldable component 21 and the lower moldable component 31 are made from a different material than the upper frame 22 and the lower frame 32. The upper frame 22 and the lower frame 32 are constructed from a more stable material compared to the upper moldable component 21 and the lower moldable component 31. This material is characterized by its high-temperature stability, allowing it to retain its original shape after molding, thereby providing structural support to the mandibular advancement device 1. A required deformation temperature of the upper moldable component 21, upon heating, is lower than that of the upper frame 22, and a required deformation temperature of the lower moldable component 31, upon heating, is lower than that of the lower frame 32. The upper moldable component 21 and the lower moldable component 31 are made from a moldable material, with a required deformation temperature upon heating being less than 100 degrees Celsius. The upper frame 22 and the lower frame 32 are made from a material with high-temperature stability, with a required deformation temperature upon heating being more than 100 degrees Celsius. In some other cases, the upper tray assembly 2 and the lower tray assembly 3 can also be made from a single material using a special manufacturing process to achieve different levels of material rigidity. The upper moldable component 21 and the lower moldable component 31 require materials with good biocompatibility, and they must be soft and comfortable to avoid causing damage to the oral cavity, making them suitable for long-term wear. Therefore, the upper moldable component 21 and the lower moldable component 31 are made from medical-grade moldable materials, typically consisting of elastic materials such as silicone rubber, polyurethane (PU), polyvinyl alcohol (PVA), ethylene-vinyl acetate (EVA), or similar materials.

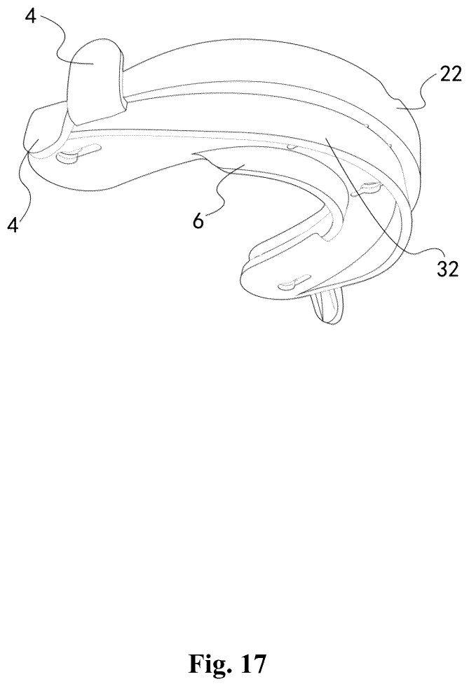

The upper frame 22 and the lower frame 32 are configured to engage and support the upper moldable component 21 and the lower moldable component 31. The upper frame 22 and the lower frame 32 have side portions positioned near the cheeks during use, and the mandibular advancement device has a terminal point 5 away from the labial side. The terminal point 5 refers to the farthest point from the labial side when the mandibular advancement device 1 is positioned in the patient's mouth. Additionally, the upper frame 22 and the lower frame 32 each have a bottom wall 7 that is positioned opposite the occlusal surfaces of the teeth during biting. At least part of the bottom wall 7 of the upper frame 22 is configured to contact the bottom wall 7 of the lower frame 32, as shown in FIG. 17. In some cases, the upper frame 22 and the lower frame 32 have at least one protrusion on the bottom wall 7 that is configured to provide a breathing channel for the patient when the mouth is closed. When wearing the mandibular advancement device 1, the device does not completely close, allowing external airflow to enter the patient's mouth through the channel, ensuring that the airflow within the oral cavity is not restricted. This design also accommodates patients who are accustomed to breathing through their mouths during sleep, reducing the risk of suffocation.

In certain cases, the upper frame 22 and the lower frame 32 are made from materials with two different levels of hardness (i.e., a first material and a second material), where the hardness of the material of the upper frame 22 is at least partially greater than the hardness of the material of the upper moldable component 21, and the hardness of the material of the lower frame 32 is at least partially greater than the hardness of the material of the lower moldable component 31. Specifically, the first material has a lower hardness than the second material. The first material is relatively soft and is configured to encase the second material due to its comfortable properties. The second material is harder and is configured to withstand greater pressure, ensuring that the upper frame 22 and the lower frame 32 do not deform or get damaged during wear and biting. The first material includes, but is not limited to, silicone rubber, polyvinyl alcohol (PVA), polyurethane (PU), ethylene-vinyl acetate (EVA), thermoplastic polyolefin (TPO), thermoplastic elastomer (TPE), thermoplastic rubber (TPR), polylactic acid (PLA), and poly(lactic-co-glycolic acid) (PLGA). The second material includes, but is not limited to, polycarbonate (PC), polypropylene (PP), acrylonitrile butadiene styrene (ABS), polyethylene (PE), polymethyl methacrylate (PMMA), polyurethane (PU), stainless steel, titanium alloy, nitinol (nickel-titanium alloy), and aluminum alloy. The first and second materials can be fixed together using various methods, including but not limited to crimping (applying pressure to bond them tightly), overmolding, snap-fit connections (where snaps are formed on the first material 4 and the second material 5 for engagement), bonding (e.g., using adhesives), thermal welding (heating the first material 4 until it melts and then cooling it onto the second material 5 to fix it), or by using additional fastening components such as screws or hooks.

The primary use of the mandibular advancement device 1 involves joining the upper moldable component 21 with the upper frame 22 to form the upper tray assembly 2 and joining the lower moldable component 31 with the lower frame 32 to form the lower tray assembly 3. By fixing the relative position between the upper tray assembly 2 and the lower tray assembly 3, the patient's mandible is pulled away from the lingual side to open the airway.

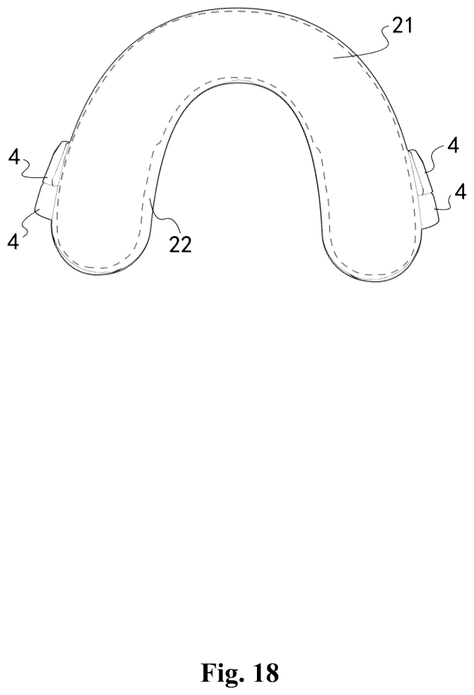

To further enhance patient comfort, the dimensions of the upper moldable component 21 and the lower moldable component 31 are specifically defined in relation to the upper frame 22 and the lower frame 32. The design ensures that the upper moldable component 21 and the lower moldable component 31 each at least partially cover the tops of the outer walls 6 of the upper frame 22 and the lower frame 32, respectively. Additionally, the projected area of the upper moldable component 21 on a horizontal plane is larger than that of the upper frame 22, and the projected area of the lower moldable component 31 on a horizontal plane is larger than that of the lower frame 32 (as shown in FIG. 18). The parts of the mandibular advancement device 1 that come into contact with the patient's oral cavity use flexible materials to minimize interference, irritation, and abrasion to the oral tissues. This approach also provides greater flexibility for the patient. Since the distance from the lips to the molar area varies among different patients, an overly long mandibular advancement device 1 could affect biting function or cause tissue abrasion. By allowing the device to be trimmed according to individual needs and preferences, the comfort and customization of the mandibular advancement device 1 are improved. The design also ensures that the height at the middle portion of the upper frame 22 and the lower frame 32 is greater than the height at the terminal point 5 of the device 1. This ergonomic design is based on the fact that the central space in the mouth is larger than the space at the sides, which helps prevent irritation and abrasion to oral tissues while enhancing the patient's comfort during treatment.

Detailed embodiments are presented below to elucidate the configurations of mandibular advancement device 1 provided in this disclosure.

### Embodiment 1

The mandibular advancement device 1 of this embodiment includes an upper tray assembly 2, configured to be positioned adjacent to the patient's upper dental arch when placed in the oral cavity, and a lower tray assembly 3, configured to be positioned adjacent to the patient's lower dental arch when placed in the oral cavity. The upper tray assembly 2 includes an upper moldable component 21 and an upper frame 22. The upper moldable component 21 and the upper frame 22 are configured to interconnect during use and to form at least one press fit when the patient bites down, and a deformation temperature of the upper moldable component 21, upon heating, is lower than that of the upper frame 22. Similarly, the lower tray assembly 3 includes a lower moldable component 31 and a lower frame 32. The lower moldable component 31 and the lower frame 32 are configured to interconnect during use and to form at least one press fit when the patient bites down, and a deformation temperature of the lower moldable component 31, upon heating, is lower than that of the lower frame 32. The upper frame 22 and the lower frame 32 each have side portions configured to be near the cheeks during use, and at least one of these side portions includes at least one protrusion 4. The protrusion 4 is configured to allow the mouth to open and close while maintaining the front-to-back positional relationship of the upper frame 22 and the lower frame 32.

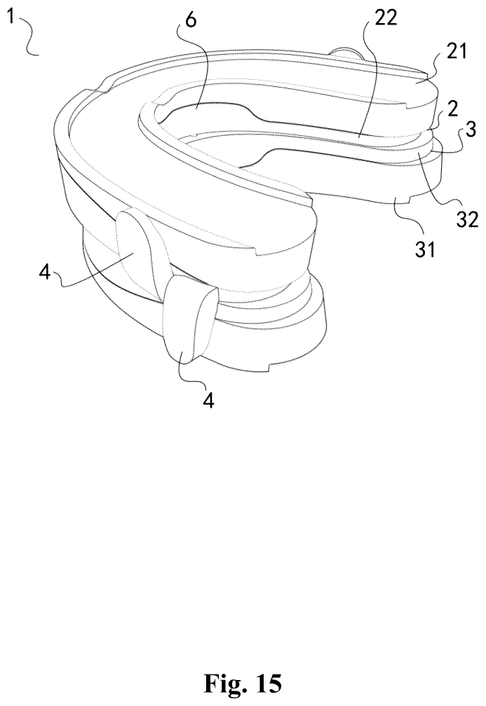

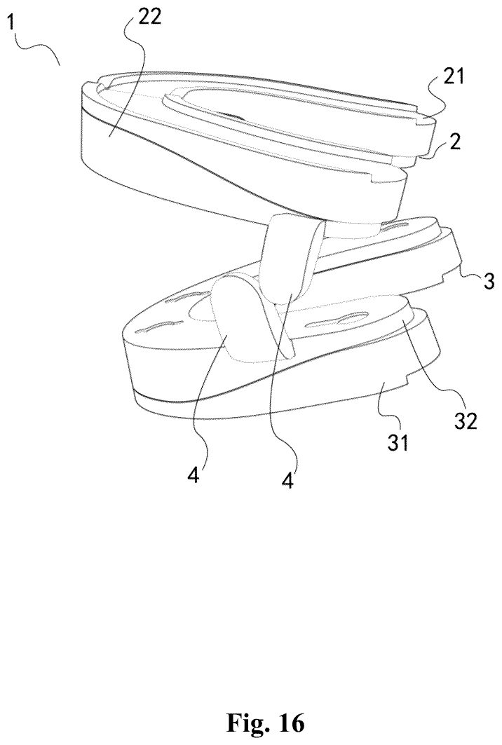

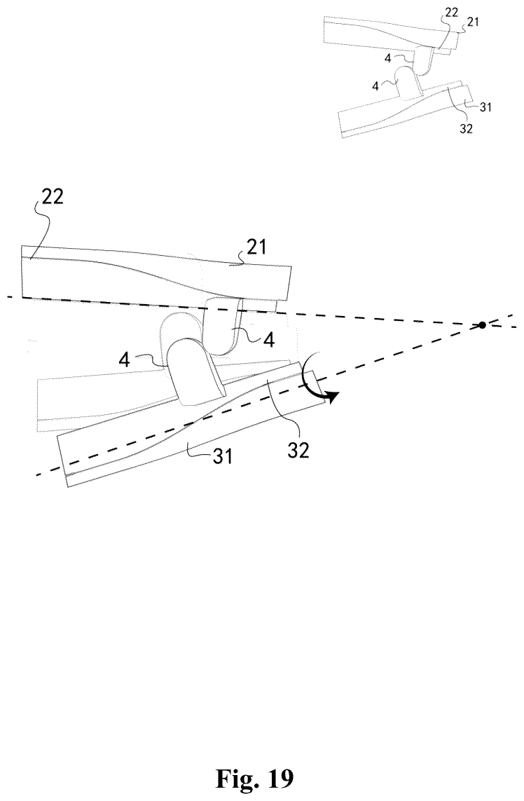

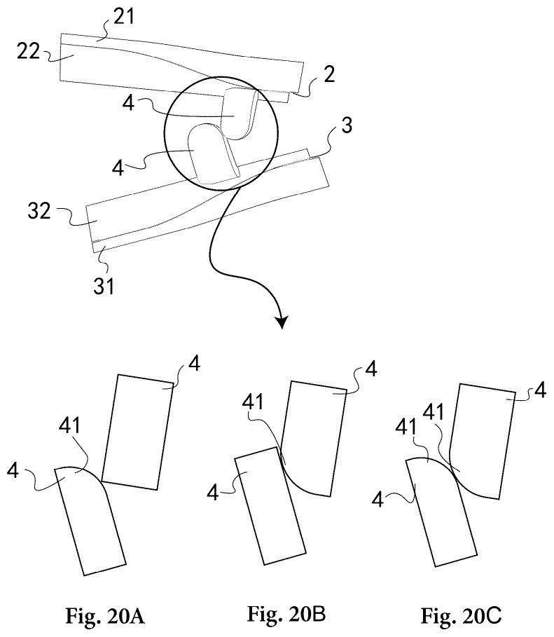

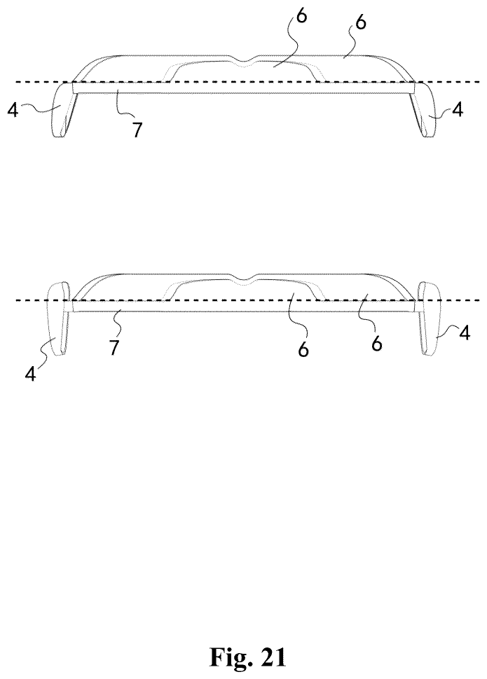

Specifically, the thermoplastic mandibular advancement device 1 of this disclosure allows the patient to open and close their mouth during treatment. When the mandibular advancement device 1 is positioned in the patient's oral cavity, the upper tray assembly 2 and the lower tray assembly 3 can be separated. In other words, the bottom wall 7 of the upper tray assembly 2 is spaced apart from the bottom wall 7 of the lower tray assembly 3, without making contact, while the lower tray assembly 3 remains fixed in position relative to the upper tray assembly 2. The connection between the upper tray assembly 2 and the lower tray assembly 3 is established by the upper frame 22 connecting to the lower frame 32. The fixed positional relationship and connection between the upper frame 22 and the lower frame 32 are specifically achieved through the at least one protrusion 4, which assists in maintaining contact between the upper frame 22 and the lower frame 32 during use, preventing them from separating. In both the open and closed states of the upper tray assembly 2 and the lower tray assembly 3, the protrusion 4 of the lower frame 32 is positioned closer to the front side near the lips compared to the protrusion 4 of the upper frame 22. When the mandible applies force to the lower tray assembly 3, the protrusion 4 of the upper frame 22 acts as a stop against the protrusion 4 of the lower frame 32. The at least one protrusion 4 on the upper frame 22 and the lower frame 32 have a specific length to ensure that when the upper tray assembly 2 and the lower tray assembly 3 are in the open state, the upper frame 22 and the lower frame 32 remain in contact and fixed through the at least one protrusion 4 (as shown in FIGS. 15 and 16). The protrusion 4 of the upper frame 22 is connected to and extends downward from the outer wall 6 of the upper frame 22, while the protrusion 4 of the lower frame 32 is connected to and extends upward from the outer wall 6 of the lower frame 32. One side of the protrusion 4 of the upper frame 22 is always in contact with one side of the protrusion 4 of the lower frame 32 (as shown in FIG. 19, where the upper frame and the lower frame rotate according to the patient's mandibular movement or rotation point). The protrusion 4 forms at least one arc 41 (as shown in FIGS. 20A, 20B and 20C). This arc 41 can be present on one or both sides of the protrusion of the upper frame, on one or both sides of the protrusion of the lower frame, or on both the upper frame and the lower frame. The arc 41 is crucial during the gradual opening process of the upper tray assembly 2 and the lower tray assembly 3. The design of the arc 41 ensures a smooth transition as the device opens and closes, reducing friction and resistance, preventing the structure from jamming, and thus providing a more stable and comfortable experience. This design ensures continuity and effectiveness in the treatment process. The connection of the protrusion 4 to the frame can include the form that the upper frame 22 and the lower frame 32, each have a bottom wall 7 positioned opposite the occlusal surface of the teeth during wear. The connection point of the protrusion 4 with the upper frame 22 and/or the lower frame 32 is flush with the bottom wall 7 of the upper frame 22 and/or the lower frame 32 (as shown in FIG. 21). The protrusion 4 may also be connected to the outer wall 6 of the upper frame 22 and/or the lower frame 32, in which case the connection point is not flush with the bottom wall 7. The protrusion 4 can take various forms. In this embodiment, the protrusion 4 is configured in an L-shape, where the at least one protrusion 4 extends from the connection points with the upper frame 22 and the lower frame 32, forming a complete protrusion 4, with the connection points being the tops of the at least one protrusion. This configuration reduces the overall size of the mandibular advancement device 1, enhances the comfort for the patient when wearing the device, and reduces the risk of the protrusion 4 causing abrasion in the patient's oral cavity. The protrusion 4 of the upper frame 22 and the lower frame 32 is typically integrally formed with the upper frame 22 and the lower frame 32, and is made from at least one of the same materials as the upper frame 22 and the lower frame 32. In some special cases, the protrusion 4 may not be integrally formed with the upper frame 22 and the lower frame 32, and the material of the protrusion 4 may differ from that of the upper frame 22 and the lower frame 32.

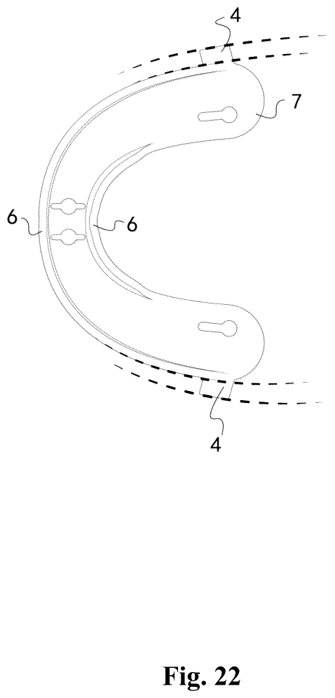

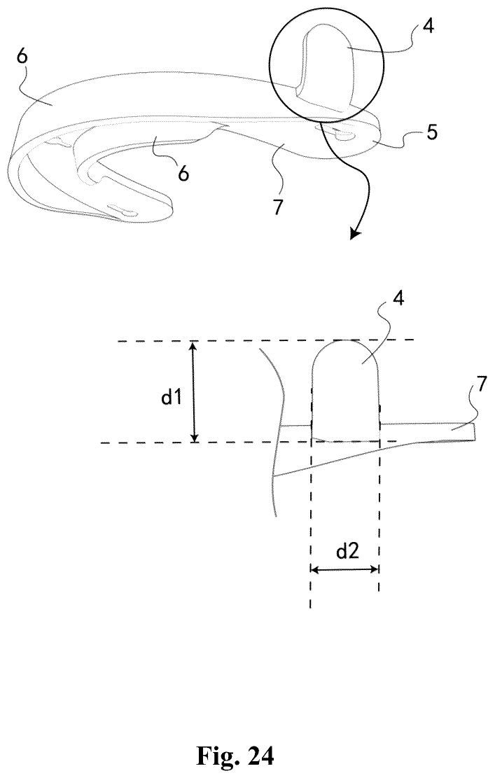

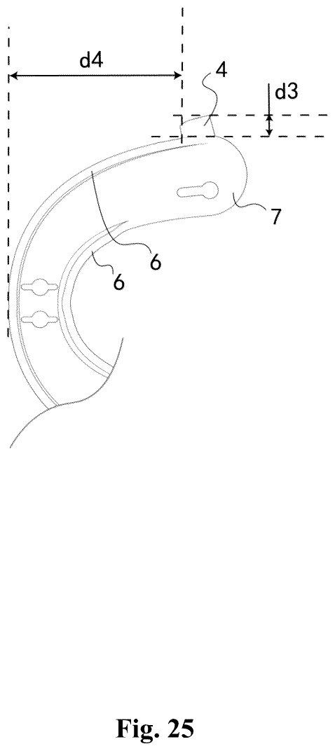

To ensure that the protrusion 4 effectively limits the position of the upper frame 22 and the lower frame 32, the distance between the protrusion 4 of the upper frame 22 and the terminal point 5 is shorter than the distance between the protrusion 4 of the lower frame 32 and the terminal point 5. Additionally, at least a portion of the protrusion 4 is positioned nearer the cheek than the outer wall 6 during use, and at least a portion of the protrusion 4 of the upper frame 22 is positioned nearer the cheek than the protrusion 4 of the lower frame 32. This design ensures that the protrusion 4 of the upper frame 22 effectively limits the movement of the protrusion 4 of the lower frame 32. Furthermore, to minimize the negative impact of the mandibular advancement device 1 on the patient's oral cavity and reduce the sensation of a foreign object, the upper frame 22 is designed with an outer wall 6, and the protrusion 4 of the upper frame 22 is shaped to conform to the curvature of the outer wall 6 of the upper frame 22. Similarly, the lower frame 32 has an outer wall 6, and the protrusion 4 of the lower frame 32 is shaped to conform to the curvature of the outer wall 6 of the lower frame 32 (as shown in FIG. 22). The dimensions of the protrusion 4 are carefully defined. The height of the protrusion 4 is set within a range of 1.7 mm to 22.5 mm (as shown in FIG. 24, where 1.7 mm≤d1≤22.5 mm), ensuring that it allows for a wide mouth-opening angle without causing discomfort or pressing against the patient's oral tissues. In some cases, the width of the protrusion is also specified to ensure stability and effectiveness. The width is set to be equal to or greater than 4 mm (as shown in FIG. 24, where d2≥4 mm). Additionally, the distance between the protrusion 4 and other parts of the frame in the top view is less than or equal to 16.5 mm (as shown in FIG. 25, where d3≤16.5 mm) to maintain the restrictive effects between the at least one protrusion of the upper frame 22 and the lower frame 32, preventing them from pressing against the patient's oral tissues. Moreover, the upper frame 22 and the lower frame 32 are designed with front ends near the labial side during use, and the distance between the protrusion 4 and the front end is set to be equal to or greater than 10 mm (as shown in FIG. 25, where d4≥10 mm). This distance is crucial as it influences the height of the frames and the opening angle between the upper tray assembly 2 and the lower tray assembly 3. Designing the position, size, and other characteristics of the protrusion 4 involves careful consideration to ensure optimal comfort and functionality.

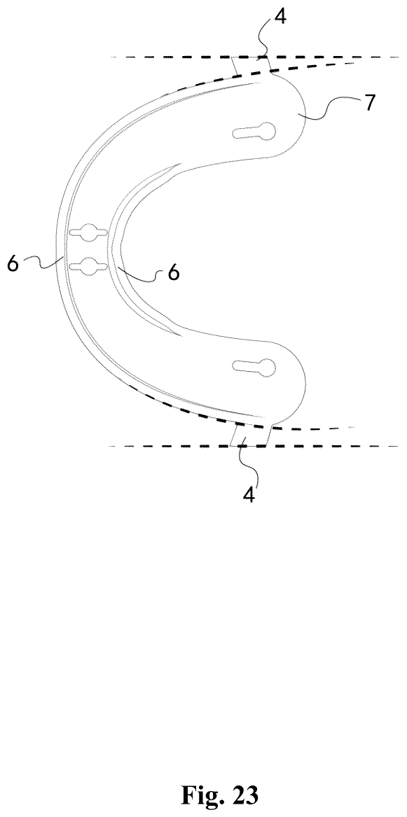

In some other embodiments, the curvature of the protrusion 4 on the upper frame 22 and/or the lower frame 32 may differ from the curvature of the frame at the location of the protrusion 4 (as shown in FIG. 23).

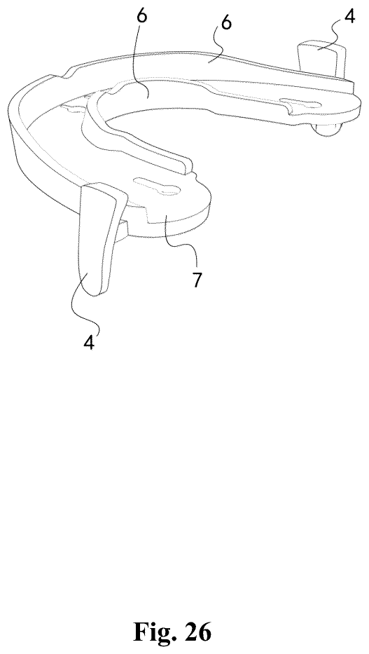

In yet other embodiments, the protrusion 4 may take on shapes other than an L-shape, such as a T-shape, H-shape, or other configurations (as shown in FIG. 26, where the protrusion 4 is illustrated in a T-shaped form).

### Embodiment 2

The mandibular advancement device 1 in this embodiment includes an upper tray assembly 2, configured to be positioned adjacent to the patient's upper dental arch when placed in the oral cavity, and a lower tray assembly 3, configured to be positioned adjacent to the patient's lower dental arch when placed in the oral cavity. The upper tray assembly 2 includes an upper moldable component 21 and an upper frame 22. The upper moldable component 21 and the upper frame 22 are configured to interconnect during use and to form at least one press fit when the patient bites down, and a deformation temperature of the upper moldable component 21, upon heating, is lower than that of the upper frame 22. Similarly, the lower tray assembly 3 includes a lower moldable component 31 and a lower frame 32. The lower moldable component 31 and the lower frame 32 are configured to interconnect during use and to form at least one press fit when the patient bites down, and a deformation temperature of the lower moldable component 31, upon heating, is lower than that of the lower frame 32. The upper frame 22 and the lower frame 32 each have side portions positioned near the cheeks during use. At least one of these side portions on either the upper frame 22 or the lower frame 32, or both, includes at least one protrusion 4. The protrusion 4 is configured to allow the patient to open and close their mouth while maintaining the front-to-back positional relationship between the upper frame 22 and the lower frame 32 during use.

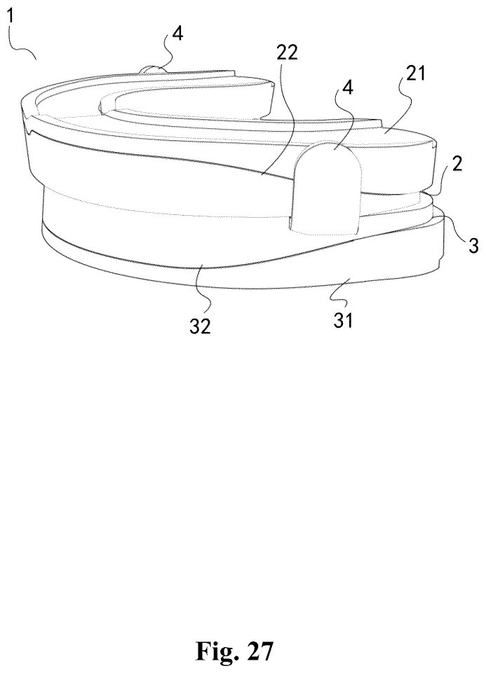

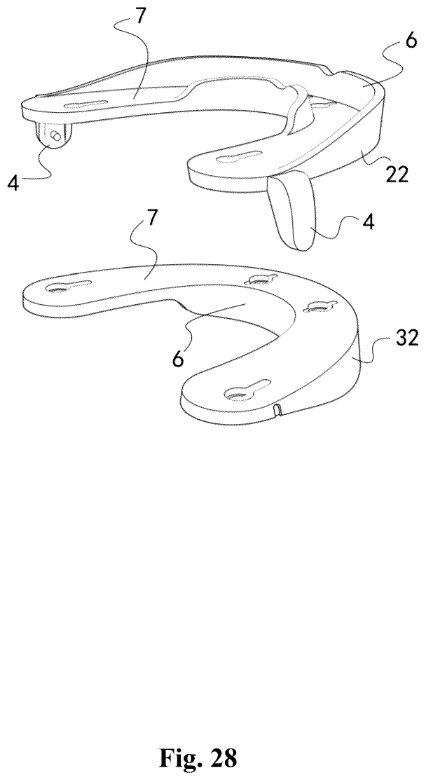

The difference between this embodiment and Embodiment 1 is that the mandibular advancement device 1 has a protrusion 4 on either the upper frame 22 or the lower frame 32, but not both (as shown in FIGS. 27 and 28). In some variations, one of the frames (either the upper frame 22 or the lower frame 32) has a protrusion 4, while the other frame may have a corresponding structure, such as a groove or snap-fit mechanism, that engages with the protrusion 4. These structures must ensure that the upper frame 22 and the lower frame 32 can be securely fixed in both the open and closed positions. This single-sided protrusion 4 design not only maintains the reliability of the mandibular advancement device 1 but also simplifies the device's structure and manufacturing process, thereby reducing production costs to some extent. Additionally, this design of the single-sided protrusion 4 takes into account the patient's comfort during wear, ensuring that the device can adapt to various movements within the patient's mouth without exerting unnecessary pressure or causing discomfort to the teeth or oral structures.

In other embodiments, both the upper frame 22 and the lower frame 32 have at least one protrusion 4, with the at least one protrusion on the upper frame 22 and the lower frame 32 being of different forms.

Furthermore, the technical features described in the above embodiments can be combined as needed to create a mandibular advancement device 1 that includes all or some of the mentioned features.

The implementation of a mandibular advancement device provided by this disclosure has at least the following beneficial effects:
1. Traditional thermoplastic mandibular advancement devices, based on general feedback, still have many areas for improvement. In particular, most thermoplastic devices have a fixed structure that does not accommodate the need for patients to open their mouths to breathe during treatment. Although some mandibular advancement devices have added at least one protrusion on the bottom walls of the upper frame and/or the lower frames to create a breathing passage, this method does not allow patients to freely control the range of mouth opening and closing. This also can be inconvenient for patients who prefer to keep their mouths closed during treatment. To overcome this limitation, this disclosure introduces an innovative and improved design for the mandibular advancement device, enabling it to be adjustable to any angle within a certain range for both open and closed mouth conditions. This is achieved by placing at least one protrusion on at least one side portion near the cheeks on the upper frame and/or the lower frames, replacing the traditional form of snap-fitting or hinge connections at the bottom walls of the frames. This design not only enhances the user experience by allowing patients to naturally perform daily activities while wearing the device, but also significantly improves treatment compliance and patient satisfaction.
2. This disclosure, through careful design improvements, offers a mandibular advancement device that is more comfortable for the patient. Extensive research has been conducted to address various challenges, resulting in a device that enhances comfort during use. Since the device needs to fit closely with the human body, it requires careful attention to ergonomics and a design process informed by extensive human data, making it a complex and challenging project. For instance, the device must stay securely in place against the patient's upper dental arch during use. The height and width of the at least one protrusion are specifically designed to allow the patient to comfortably open and close their mouth with minimal impact. The design of the at least one protrusion is such that they continuously restrict the patient's lower jaw during the opening and closing of the mouth (i.e., maintaining the effect of mandibular advancement), ensuring the upper frame and the lower frame of the device stay connected without separating from each other while the mouth moves. These improvements were developed through targeted design solutions and extensive testing. The details of the upper frame and the lower frame, as well as the moldable components have been optimized to ensure they fit the dental arches more closely. The upper tray assembly, in particular, is designed to fit snugly with the upper dental arch and stay in place, preventing detachment due to gravity even when the mouth is open. The height of the at least one protrusion is set between 1.7 mm and 22.5 mm to allow for a wide mouth opening without pressing against the patient's oral structure. The distance between the at least one protrusion and the front ends is set to be more than 10 mm. Placing the at least one protrusion closer to the terminal point of the device can increase the opening angle between the upper frame and the lower frame but may also increase the chance of contact with the patient's oral tissues, possibly causing discomfort. Therefore, a balance is needed between these factors. The at least one protrusion on the upper frame and/or the lower frames needs to engage at any angle within a certain range, so they are designed with a slight outward offset. This offset helps ensure the effectiveness of the treatment and improves patient comfort. Additionally, the contact point between the at least one protrusion on the upper frame and the lower frame includes at least one arc. This design ensures smooth operation during the opening and closing of the mouth, reducing friction and avoiding jamming. These design features are not just simple structural choices; they are the result of precise calculations and optimizations. To achieve the best results, extensive experimental testing and data recording were necessary. Through repeated trials and fine adjustments, the shapes and dimensions of the at least one protrusion were gradually refined. Extensive data analysis and feedback were used to optimize the design of the at least one protrusion and other aspects, ensuring that every detail has been rigorously tested and optimized to guarantee patient comfort and the durability of the device.
3. This disclosure offers a more cost-effective alternative that achieves the same results as customized mandibular advancement devices. In the current market, only customized devices can effectively achieve the desired mouth opening and closing function. These custom devices are tailored under the guidance of professional physicians, ensuring a perfect fit with the patient's teeth and oral structure, which provides a high level of comfort. However, because they are custom-made, they require advanced manufacturing equipment and a fully digital manufacturing process, making them expensive—often costing thousands of dollars, which can be a significant financial burden for patients. In contrast, this disclosure integrates at least one protrusion into a non-custom mandibular advancement device, along with the use of upper and lower moldable components and frames, and precise control over various parameters of the device. This design allows the device to achieve the same mouth opening and closing effect as custom devices. It is a more suitable option for patients with limited budgets, offering an affordable treatment solution without compromising the device's effectiveness or comfort. This improvement makes the device accessible to a wider range of patients, achieving a better balance between personalization and cost-effectiveness. Additionally, this mandibular advancement device can be produced quickly, reducing the waiting time associated with custom devices, making it a better choice for patients seeking faster treatment.
4. The mandibular advancement device provided by this disclosure not only expands the scenarios in which it can be used, enhancing patient comfort, but also makes treatment more stable and extends the product's lifespan. a. The mandibular advancement device provided by this disclosure meets various needs in a patient's daily life, allowing them to engage in activities such as speaking and drinking while wearing the device, without the need to use the upper and lower dental arches to forcefully clamp and hold the device in place in the mouth. Most patients who require mandibular advancement devices suffer from snoring, particularly those who snore due to throat issues, where the mouth involuntarily opens and closes with each breath. Traditional devices with rigid structures make it difficult to breathe naturally and perform other oral activities while wearing them. The mandibular advancement device provided by this disclosure reduces the psychological burden on patients, adapts to the needs of different individuals, and offers a more ideal treatment option. b. The design of this mandibular advancement device carefully considers the complexity of the oral structure, ensuring comfort through precise calculations. Devices with rigid fixation can restrict oral movement, potentially causing discomfort or even pain during use. The inclusion of at least one protrusion in this design allows for a certain degree of freedom in oral movement, significantly improving comfort. c. Additionally, the upper and lower moldable components are made of flexible materials, further enhancing the safety and comfort of the device for the teeth. Typically, the main body of a custom-made mandibular advancement device (the part that directly contacts the teeth) is made from a single rigid material. In contrast, this disclosure features flexible materials that come into contact with the inside of the mouth, avoiding pressing against the oral tissues and reducing the sensation of a foreign object in the mouth during use. Moreover, the flexible material provides cushioning in the event of an accidental impact, offering protection to the teeth and making this a safer mandibular advancement device. d. The at least one protrusion in the mandibular advancement device provided by this disclosure allows it to be used in a variety of situations, increasing the product's stability and making it less likely to dislodge during use. As a result, patients can use this device for longer periods, reducing interruptions in treatment caused by device movement or other issues, and ensuring better adherence to the treatment regimen. e. The at least one protrusion also ensures a stable fit between the upper and lower tray assemblies and the patient's upper and lower dental arches, even when the mouth is open. This reduces fatigue from continuously clenching the device, meaning patients do not need to replace the device frequently. This feature is highly beneficial, contributing to increased patient satisfaction.

The technical features of the above embodiments can be freely combined. For brevity, not all possible combinations of these features are described here. However, as long as the combinations do not introduce contradictions, they should be considered within the scope outlined by this disclosure.

The embodiments described above represent only a few of the possible implementations of the disclosure. Although the descriptions are specific and detailed, they should not be understood as limiting the scope of this disclosure. It should be noted that for one skilled in the art, various modifications and improvements can be made without departing from the concept of the disclosure, and these are also considered within the scope of protection of this disclosure. Therefore, the scope of protection for this disclosure should be determined by the appended claims.

It must be noted that as used herein and in the appended claims, the singular forms “a”, “an”, and “the” include their plural equivalents, unless the context clearly dictates otherwise.

1. A mandibular advancement device comprising:
an upper tray assembly, configured to be positioned adjacent to an upper dental arch of a patient when placed in an oral cavity of the patient,
wherein the upper tray assembly includes an upper moldable component and an upper frame, the upper moldable component and the upper frame are configured to interconnect during use and to form at least one press fit when the patient bites down, and a required deformation temperature of the upper moldable component, upon heating, is lower than that of the upper frame;
a lower tray assembly, configured to be positioned adjacent to a lower dental arch of the patient when placed in the oral cavity,
wherein the lower tray assembly includes a lower moldable component and a lower frame, the lower moldable component and the lower frame are configured to interconnect during use and to form at least one press fit when the patient bites down, and a required deformation temperature of the lower moldable component, upon heating, is lower than that of the lower frame;
at least one protrusion disposed on the upper frame; and
at least one protrusion disposed on the lower frame,
wherein the upper frame and the lower frame each have side portions configured to be near cheeks of the patient during use, the protrusion disposed on the upper frame is attached to at least one of the side portions of the upper frame, and the protrusion disposed on the lower frame is attached to at least one of the side portions of the lower frame, and the protrusion of the upper frame is immovable relative to the upper frame,
wherein in an upper-to-lower frame direction, the protrusion disposed on the upper frame extends downward, and the protrusion disposed on the lower frame extends upward,
the protrusion disposed on the upper frame and the protrusion disposed on the lower frame are configured to contact with each other via a contact surface when in use,
the protrusion disposed on the upper frame and the protrusion disposed on the lower frame are configured to allow a mouth of the patient to open and close while maintaining a front-to-back positional relationship of the upper frame and the lower frame.

2. The mandibular advancement device according to claim 1, wherein the upper moldable component and the lower moldable component are configured in an arched shape, which is at least one shape selected from the group consisting of an oval arch, a conical arch, and a square arch.

3. The mandibular advancement device according to claim 1, wherein the upper frame and the lower frame each have a bottom wall that is configured to be opposite an occlusal surface of teeth when the patient bites down, and at least part of the bottom wall of the upper frame is configured to contact the bottom wall of the lower frame.

4. The mandibular advancement device according to claim 1, wherein the upper moldable component and the lower moldable component include a flexible thermoplastic material.

5. The mandibular advancement device according to claim 1, wherein a hardness of a material of the upper frame is at least partially greater than a hardness of a material of the upper moldable component, and a hardness of a material of the lower frame is at least partially greater than a hardness of a material of the lower moldable component.

6. The mandibular advancement device according to claim 1, wherein the upper tray assembly and the lower tray assembly each have a contour that is higher at a middle and lower at sides nearer the cheeks during use.

7. A mandibular advancement device comprising:
an upper tray assembly, having an arched contour configured to conform to a curvature of an upper dental arch of a patient,
wherein the upper tray assembly includes an upper moldable component and an upper frame, the upper moldable component and the upper frame are configured to interconnect during use, and a required deformation temperature of the upper moldable component, upon heating, is lower than that of the upper frame;
a lower tray assembly, having an arched contour configured to conform to a curvature of a lower dental arch of the patient,
wherein the lower tray assembly includes a lower moldable component and a lower frame, the lower moldable component and the lower frame are configured to interconnect during use, and a required deformation temperature of the lower moldable component, upon heating, is lower than that of the lower frame;
at least one protrusion disposed on the upper frame; and
at least one protrusion disposed on the lower frame,
wherein the upper frame and the lower frame each have side portions configured to be near cheeks of the patient during use, the protrusion disposed on the upper frame is attached to at least one of the side portions of the upper frame, and the protrusion disposed on the lower frame is attached to at least one of the side portions of the lower frame, and the protrusion of the upper frame is immovable relative to the protrusion of the lower frame.
wherein in an upper-to-lower frame direction, the protrusion disposed on the upper frame extends downward, and the protrusion disposed on the lower frame extends upward,
the protrusion disposed on the upper frame and the protrusion disposed on the lower frame are configured to contact with each other via a contact surface when in use,
the protrusion disposed on the upper frame and the protrusion disposed on the lower frame are configured to allow a mouth of the patient to open and close while maintaining a front-to-back positional relationship of the upper frame and the lower frame, and
the protrusion disposed on the upper frame and the protrusion disposed on the lower frame have one or more of the following characteristics:
(a) a height of the protrusion is between 1.7 mm to 22.5 mm;
(b) a distance between the protrusion disposed on the upper frame and other parts of the upper frame or a distance between the protrusion disposed on the lower frame and other parts of the lower frame is less than 16.5 mm in a top view; and
(c) the upper frame and the lower frame have front ends configured to be near labial sides of the patient during use, and a distance between the at least one protrusion and the front ends is greater than 10 mm.

8. The mandibular advancement device according to claim 7, wherein the upper frame has at least one groove to accommodate the upper moldable component.

9. The mandibular advancement device according to claim 7, wherein the lower frame has at least one groove to accommodate the lower moldable component.

10. The mandibular advancement device according to claim 7, wherein the upper moldable component and the lower moldable component are configured to soften when heated in hot water such that the upper moldable component and the lower moldable component are adjusted and molded to a shape that is configured to conform to the upper dental arch and the lower dental arch, respectively, when the patient bites down.

11. The mandibular advancement device according to claim 10, wherein the upper moldable component and the lower moldable component are made of a moldable material, the required deformation temperature upon heating being less than 100 degrees Celsius.

12. The mandibular advancement device according to claim 11, wherein the upper frame and the lower frame are made of a material with high thermal stability, the required deformation temperature upon heating being greater than 100 degrees Celsius.

13. A mandibular advancement device comprising:
an upper tray assembly, configured to be positioned adjacent to an upper dental arch of a patient when placed in an oral cavity of the patient,
wherein the upper tray assembly includes an upper moldable component and an upper frame, the upper moldable component and the upper frame are configured to interconnect during use and to form at least one press fit when the patient bites down, and a required deformation temperature of the upper moldable component, upon heating, is lower than that of the upper frame;
a lower tray assembly, configured to be positioned adjacent to a lower dental arch of the patient when placed in the oral cavity,
wherein the lower tray assembly includes a lower moldable component and a lower frame, the lower moldable component and the lower frame are configured to interconnect during use and to form at least one press fit when the patient bites down, and a required deformation temperature of the lower moldable component, upon heating, is lower than that of the lower frame;
at least one protrusion disposed on the upper frame; and
at least one protrusion disposed on the lower frame,
wherein the upper frame and the lower frame each have side portions configured to be near cheeks of the patient during use, the protrusion disposed on the upper frame is attached to at least one of the side portions of the upper frame, and the protrusion disposed on the lower frame is attached to at least one of the side portions of the lower frame, and the protrusion of the upper frame is immovable relative to the upper frame
wherein in an upper-to-lower frame direction, the protrusion disposed on an upper frame extends downward, and the at least protrusion disposed on a lower frame extends upward,
the protrusion disposed on the upper frame and the protrusion disposed on the lower frame are configured to contact with each other via a contact surface when in use,
the protrusion disposed on the upper frame and the protrusion disposed on the lower frame are configured to allow a mouth of the patient to open and close while maintaining a front-to-back positional relationship of the upper frame and the lower frame.

14. The mandibular advancement device according to claim 13, wherein the upper frame remains in contact with the lower frame without separating during use.

15. The mandibular advancement device according to claim 13, wherein the upper moldable component and the lower moldable component are made of a medical-grade moldable material.

16. The mandibular advancement device according to claim 15, wherein, when the at least one press fit is formed in the upper moldable component and the lower moldable component, the moldable components allow for adjustments so as to fine-tune the at least one press fit while retaining their deformed shape.

17. The mandibular advancement device according to claim 13, wherein the upper frame and the lower frame each have an outer wall, and the upper moldable component and the lower moldable component each at least partially cover a top of the outer wall, respectively.

18. A mandibular advancement device comprising:
an upper tray assembly, having an arched contour configured to conform to a curvature of an upper dental arch of a patient,
wherein the upper tray assembly includes an upper moldable component and an upper frame, the upper moldable component and the upper frame are configured to interconnect during use and to form at least one press fit when the patient bites down, and a required deformation temperature of the upper moldable component, upon heating, is lower than that of the upper frame;
a lower tray assembly, having an arched contour configured to conform to a curvature of a lower dental arch of the patient,
wherein the lower tray assembly includes a lower moldable component and a lower frame, the lower moldable component and the lower frame are configured to interconnect during use and to form at least one press fit when the patient bites down, and a required deformation temperature of the lower moldable component, upon heating, is lower than that of the lower frame; and
at least one protrusion disposed on the upper frame; and
at least one protrusion disposed on the lower frame,
wherein the upper frame and the lower frame each have side portions configured to be near cheeks of the patient during use, the protrusion disposed on the upper frame is attached to at least one of the side portions of the upper frame, and the protrusion disposed on the lower frame is attached to at least one of the side portions of the lower frame, and the protrusion of the upper frame is immovable relative to the upper frame
wherein in an upper-to-lower frame direction, the protrusion disposed on the upper frame extends downward, and the protrusion disposed on the lower frame extends upward,
the protrusion disposed on the upper frame and the protrusion disposed on the lower frame are configured to contact with each other via a contact surface when in use,
the protrusion disposed on the upper frame and the protrusion disposed on the lower frame are configured to allow a mouth of the patient to open and close while maintaining a front-to-back positional relationship of the upper frame and the lower frame, and
the mandibular advancement device has a terminal point that is configured to be away from a labial side during use, a distance between the at least one protrusion disposed on the upper frame and the terminal point being shorter than a distance between the at least one protrusion disposed on the lower frame and the terminal point.

19. The mandibular advancement device according to claim 18, wherein the upper frame has an outer wall, and the at least one protrusion disposed on the upper frame conforms to a curvature of the outer wall of the upper frame.

20. The mandibular advancement device according to claim 18, wherein the lower frame has an outer wall, and the at least one protrusion disposed on the lower frame conforms to a curvature of the outer wall of the lower frame.

21. The mandibular advancement device according to claim 18, wherein a height at a middle portion of the upper frame and a height at a middle portion of the lower frame are greater than a height at the terminal point of the mandibular advancement device.

22. The mandibular advancement device according to claim 18, wherein the upper frame and the lower frame each have a bottom wall that is configured to be opposite an occlusal surface of teeth during use, and a connection point with the upper frame of the at least one protrusion disposed on the upper frame and a connection point with the lower frame of the at least one protrusion disposed on the lower frame is flush with the bottom walls of the upper frame and the lower frame, respectively.
